(19) United States
(12) Patent Application Publication
(10) Pub. No.: US 2026/0015589 A1
(43) Pub. Date: Jan. 15, 2026

(54) COMPOSITIONS, METHODS, AND ASSOCIATED DEVICES FOR CRYOPRESERVATION OF BIOLOGICAL SPECIMENS

(71) Applicant: Ranan Gulhan Aktas, Milton, MA (US)

(72) Inventor: Ranan Gulhan Aktas, Milton, MA (US)

(21) Appl. No.: 18/992,974

(22) PCT Filed: Jul. 11, 2023
(86) PCT No.: PCT/TR2023/050674

§ 371 Date: Jan. 10, 2025

Related U.S. Application Data
(60) Provisional application No. 63/388,149, filed on Jul. 11, 2022.

(30) Foreign Application Priority Data
Jul. 11, 2022 (US) 63/388149
Jul. 11, 2023 (WO) PCT/TR2023/050674

(51) Int. Cl. C12N 5/00

(52) U.S. Cl. CPC C12N 5/525; C12N 5/526; C12N 5/545

(57) ABSTRACT

The present invention relates to the compositions, methods, and associated devices (1) for the cryopreservation of biological specimens that enables to freeze and thaw the entire specimen by straightforwardly protecting its three-dimensional architecture. The invention comprises compositions including reagents to protect the specimen and preserve the physiological conditions. The methods comprise fewer steps excluding the need for other solutions used in conventional methods. The methods also eliminate stressful steps for biological specimens in conventional methods including harvesting, pipetting, centrifuging, and transferring. With this invention, the entire content of the specimen can be frozen, stored long-term in a single cell culture vessel with one of the associated devices (1) described here, and then thawed while still in the device (1). The biological specimens frozen by that method render high post-thawing viability. Moreover, the invention reduces cryoprotectant-related toxic events, human errors, and contamination risk.

### CROSS REFERENCE TO RELATED APPLICATION

This disclosure claims the benefit of U.S. nonprovisional application No. US 2024/0200004 A1. entitled “Multipurpose Container for Biological Materials and Methods, filed Jul. 21, 2023, which is incorporated herein by reference in their entirety.

### TECHNICAL FIELD

The present invention relates to compositions, methods, and associated devices for cryopreservation of biological specimens, e.g., organoids, spheroids, cells, cell lines, multicellular samples, living organisms, artificial or natural tissues, harvested organs and pathological samples, in a simplified, standardized, and straightforward way.

### BACKGROUND OF THE INVENTION

Methodologies related to cryopreservation of biological specimens are commonly used for research and therapy if the biological specimens are required to be stored for the long term.

Cryopreservation technologies of single dispersed cells, cell lines, and embryos has already been well established. Two principal methodologies, ‘slow freezing’ and ‘vitrification,’ are used worldwide. In ‘slow freezing,’ which is probably still the most used method, the cells are treated with cryoprotectants and cooled down gradually to −196° C. Freezing cells slowly is essential to prevent intracellular ice formation and can be achieved using a freezing rate of 1° C./minute. On the other hand, vitrification is an ultra-rapid method of cryopreservation. High concentrations of cryoprotectants are used, and rapid cooling rates are essential in that methodology. In both methodologies, the cells are grown in a specific environment like a cell culture flask, well-plate, or culture dishes. Then, the specimen is transferred to a vessel-like cryovials, cryo-loops, or cryopreservation straws-for storage in a freezer. When thawing is needed, the specimen is required to be transferred to another container, like a culture vessel, again. The specimen is centrifuged, pipetted, and treated with dissociation/harvesting solutions for those transfers. Those lengthy and complex steps cause loss and damage of the specimen. Cryopreservation of 3D growing cells are more complex and the success rate is low, about 50% in many cases. Although some papers have reported cryopreservation of multicellular structures, such as organoids and spheroids, loss and damage to the 3D structure of those specimens during conventional freezing and thawing processes is extremely high. That problem causes time, labor, and data loss. Transfers and pipetting steps make impossible to preserve entire structures in complex models, like co-culture experiments.

The thawing process is also crucial for the successful survival of the biological specimens. Once the cells have been removed from liquid nitrogen storage, the cryovial should be placed in a 37° C. water bath until the contents are thawed. The specimens are then immediately transferred to a large volume of pre-warmed medium. Some specimens are pelleted by centrifugation before being gently resuspended with a pipette and added to a cell culture flask containing pre-warmed growth medium. In another methodology, the specimen is transferred to a flask or well-plate straight after thawing, and a medium change is made the next day to remove any residual cryoprotectant. In both ways, the transferring steps and pipetting and centrifuging cause damage to those fragile structures. In the end, those stressful steps cause damage of 3D architecture of the biological specimen, loss of data related to interaction between the structures in a multicellular environment.

Cryopreservative reagents typically comprise a cryoprotectant, and a source of protein (usually serum), the growth-medium and/or diluting agents. Cryoprotectants are used to preventing ice formation, which causes freezing damage to the living specimens. Therefore, they must be able to penetrate the cells and have low toxicity. The growth-medium component can be supplemented with conditioned serum-free medium or 10% cell culture grade BSA.

Most cryopreservative reagents rely on dimethyl sulfoxide (DMSO) at concentrations from 5-20% (McLellan, M. R., and Day, J. G. (1995) Methods Mol Biol 38: 1-5). Other chemicals such as glycerol, ethylene glycol, hydroxycellulose, sucrose, maltose, and trehalose have been shown to enhance cell viability when combined with DMSO (Gulliksson, H. (2000) Transfus Med 10: 257-264).

The cells in our body are surrounded with extracellular matrix (ECM) that supports them in different conditions, provides resistancy, support development, growth and strength. The ECM comprises two main classes of macromolecules: proteoglycans and fibrous proteins (Jarvelainen et al., 2009; Schaefer & Schaefer, 2010). Collagens are the most abundant fibrous protein within the interstitial ECM and constitute up to 30% of the total protein mass of a multicellular animal. They provide tensile strength, regulate cell adhesion, support chemotaxis and migration, and direct tissue development (Rozario & DeSimone, 2010). In addition, elastin fibers provide recoil to tissues that undergo repeated stretch. Elastin stretch is crucially limited by tight association with collagen fibrils (Wise & Weiss, 2009). A third fibrous protein, fibronectin, directs the organization of the interstitial extracellular matrix and has a crucial role in mediating cell attachment and function. Fibronectin can be stretched several times over its resting length by cellular traction forces (Smith et al., 2007). Such force-dependent unfolding of fibronectin exposes cryptic integrin-binding sites within the molecule that result in pleiotropic changes in cellular behavior and implicate fibronectin as an extracellular mechano-regulator (Smith et al., 2007). Du and Betti demonstrated the cryoprotective effect of chicken collagen hydrolysate on the natural actomyosin model system (2016). Their results suggest that collagen hydrolysate can inhibit ice crystal growth, reduce protein freeze-denaturation and oxidation similarly to the commercial cryoprotectants, and result in higher protein solubility and a better gel structure after freeze-thaw cycles.

In the prior art, US2005106554 A1 relates to methods and compositions for the cryopreservation of pluripotent cells in general and human embryonic stem (ES) cells in particular. The stem cells are grown on a bottom layer of solid support matrix and subsequently covered by a top layer of solid support matrix forming a matrix-cell-matrix composition, to which an effective amount of cryopreservation media is added, prior to freezing. The methods of the invention yield cryopreserved cells that exhibit an increase in cell viability and a decrease in cell differentiation, facilitating storage, shipping and handling of embryonic stem cell stocks and lines for research and therapeutics. The invention in this document aimed cryopreservation of 2D growing stem cells. The stem cells are grown on a bottom layer of Solid Support matrix and subsequently covered by a top layer of Solid Support matrix forming a matrix-cel-matrix composition, to which an effective amount of cryopreservation media is added prior to freezing.

In addition to the proper formulation of cryopreservation media, successful cryopreservation requires that the biological specimens are collected and carefully handled from derivation to cryopreservation in a standardized manner. Furthermore, proper equipment must be in place to ensure consistency, reproducibility, and sterility. In addition, the correct choice and amount of cryoprotectant agent must be added at the proper temperature, and a controlled rate of freezing must be applied prior to a standardized method of cryogenic storage.

The other problem of the art is to provide a cryopreservation method to preserve the 3D structure of the specimen with the higher cell viability rates and prevent damage and loss of those structures while freezing and thawing. During the available methodologies for cryopreservation—with transfers between the cell culture vessels and cryovials, pipetting, and centrifuging—those susceptible and fragile 3D-growing structures are often damaged or lost. Post-thaw organoid recovery is usually less than 50% in current technologies. The results are significantly affected since the 3D structures cannot maintain the same characteristics and quality. That is why current cryopreservation technologies for 3D structures commonly face problems of reproducibility and consistency. Use of current instruments providing slow freezing are limited due to their complexity and high costs. In a prior art, a device has been developed to cryopreserve harvested mammalian tissues and living cultured tissue equivalents (04077072.9, 2005).

Taken together, cryopreservation of the biological specimens with current technologies is a complex and lengthy process. Loss and damage of specimen's cause difficulty in standardizing and integrating the conclusions. During those procedures; viability and proliferative capacity decreases. Outcomes at the end may not reflect the conditions of the biological specimens at the beginning of the process. Transplantation of the specimens after cryopreservation is also affected with those problems. The experiments' consistency, efficiency, and reproducibility are still a big problem using current methodologies. Researchers try to find out how to enhance their research capabilities through safer cryopreservation processes while preventing contamination.

Since those 3D growing cells, organoids, spheroids, tissues, and living organisms are becoming superior experimental models and being used for personalized therapies; there is an urgent need to develop technologies to protect those susceptible structures during freezing and thawing processes. Additionally, current art does not provide solutions for cryopreservation of the whole specimens including different compartments like in co-culture studies, which are excellent models to study organogenesis, disease modelling, drug discovery, and more.

Additionally, there is no current solution to secure an experiment if there is a sudden interruption of the studies due to a change of circumstances. A technology that allows the researcher to stop the experiment in an urgent situation and then continue without any loss when the conditions are favourable would be highly beneficial; since it will save time, labor, and cost. For example, because of the COVID pandemic, scientists needed to stop the experiments immediately and lose their precious materials and data. Another situation occurs when there is a need for a specialized device or reagent to halt the investigation and freeze the specimen until a new reagent or equipment arrives at the lab. There is no current solution to hold the specimen in a safe environment until that device or reagent arrives in the lab.

The present invention is accomplished in order to solve the those foregoing problems. An object of the present invention is to provide (i) compositions for cryopreservation which can be used for cryopreserving the entire biological specimens (ii) use of the cryopreservative compositions with the new simplified methods, and (iii) use of associated devices to simplify and speed up the experiment while preventing contamination risk and reducing human errors during the use of the cryopreservation media and methods described here. Compositions, methods, and associated devices in the invention may be used in any combination, together or solely.

### BRIEF SUMMARY OF THE INVENTION

The present invention relates to compositions, methods, and associated devices for cryopreservation of biological specimens which meets the mentioned requirements above, removes disadvantages in the current art and brings some additional advantages.

The present invention includes a composition complemented with a methodology and an associated device that provides successful cryopreservation of the whole structure surrounded by an environment. For example, with that invention, organoids, spheroids, tumoroids, and other 3D growing structures in the gel are frozen in this associated device without separating 3D structures from the gel. Similarly, different components of a co-culture environment can be frozen and then thawed without disturbing their living conditions in an associated device. Furthermore, the entire tissue specimens can be frozen using the cryopreservation media and methodology. In other words, the invention provides the ‘whole mount’ cryopreservation of the specimen in one device, eliminating the harvesting, transferring, pipetting, and centrifuging steps. The entire content of the biological specimen remains in its own habitat during the cryopreservation process. That allows preservation of the entire structure, minimizes loss and damage of the specimens, provides more accurate data. Furthermore, the invention reduces human errors and contamination risks.

The composition in this invention, which serves as cryopreservation media, includes cryoprotectant agents, diluting agents and accessory agents but is not limited to. The accessory agents, e.g., the components of the extracellular matrix, mimic the protective environment in the living structures in vivo and provides the protective and preservative effects for the specimen, as described.

In some embodiments, the composition is used for slow cryopreservation, and the procedure involves slow cooling, typically at a controlled freezing rate of 1 to 2° C./min. A modified rapid thawing step follows this. In some other embodiments, the composition contains a high concentration of the ingredients listed above and is used for the vitrification method.

The associated device in this invention allows performing every step—from growth to freeze—in a single environment without transferring the biological specimen. This method offers less manipulation and increases cell viability after thawing due to the minimization of the stressful steps. Furthermore, freezing those 3D structures in a high surface-to-volume ratio allows a more homogeneous cooling and thawing rate to optimize viability rates.

This invention will not only be of great use to many researchers in the biological and medical field, but it also has the potential to impact clinical practices as it can be used for cryopreservation of patient's specimens, like oocytes, sperms, ovaries, testicular tissues, and any other tissues. The invention facilitates biobanking of biological specimens since it brings simplicity to freezing, storage, handling, and thawing steps.

With that invention, the entire biological specimen remains in its natural habitat during the freezing-thawing process. It brings a new technology allowing freezing and thawing specimens while they are still in their growing environment which allows long-term preservation of the entire specimen, prevents damage and loss of the architecture of the specimen, minimizes loss of biolo data. The compositions may be added to the environment where the biological specimen is located, e.g., into a cell culture vessel or one of the associated devices (1) described here. That enables protection of entire specimen, preservation of the physiological conditions, investigation of interactions between the different compartments in a complex multicellular architecture, and provides more accurate results. For example, with the new method it is possible to freeze and thaw an entire specimen containing different compartments and complex living structures in a co-culture experiment. Organoids and spheroids also can be frozen and thawed while they are still in their own habitat, like a hydrogel or alginate capsule.

The main advantage of the compositions, methods, and associated devices to the current technologies is that they bring quick, efficient, practical, and protective solutions for freezing and thawing entire biological specimens. The invention drastically reduces the number of steps and eliminates centrifuging, harvesting, and transferring between the steps in conventional methods. The results get more accurate since the scientist eliminates those stressful steps, and the specimen stays in the same environment during the entire process. In addition, the invention decreases contamination risk, damage and loss of the biological specimen due to handling processes; reduces human errors. The associated device provides a protective environment for the specimen while it is still in its own habitat in a container during gradually freezing. In some other embodiments, the device makes it possible to grow, freeze, thaw, continue culturing, and then examine the biological specimen in a single environment.

Another advantage of this invention is the ability to stop the experiment by freezing the specimen, preserve the entire specimen for long term, and then return to the study by thawing it when needed. The cryopreserved tissue may be stored for indefinite periods of time prior to use.

The present invention includes a newly formulated compositions for cryopreservation with the methods and the associated devices. The compositions in the present invention may include accessory agents that may comprise some of extracellular matrix components, which is essential for physical scaffolding for the cellular constituents but also initiates processes required for tissue morphogenesis and homeostasis. Therefore, adding one or more extracellular matrix components to the cryopreservation media provides a more protective environment for the specimen and supports the living structures to heal after freezing and thawing. Other accessory agents may be added to the composition to provide additional preservative and protective effects during the freezing and thawing procedures of the entire specimen. The content and amount of those accessory agents may vary according to the nature and size of the specimen in addition to the design of the experiment.

The cryopreservation compositions with the methods in that invention may comprise an associated device. The associated devices create simple, and more secure storage opportunities for biological specimens in different cell culture vessels. The device eliminates the step of transferring the specimen from a cell culture vessel to a a vessel for cryostorage, e.g. cryovial, cryoloop, and others. The device may comprise biocompatible material resistant to temperature changes between +60° C. and −196° C. and allow slow gradual freezing. It is even possible to grow and examine the specimens before or after cryopreservation in the same associated device in some embodiments. These and other advantages of the various embodiments of the compositions, methods, and associated devices described herein will be readily apparent to those of skill in the art upon reading the disclosure presented herein.

The present invention is accomplished in order to solve those foregoing problems. An object of the present invention is to provide (i) compositions for cryopreservation which can be used for cryopreserving the entire biological specimens (ii) use of the cryopreservative compositions with the new simplified methods, and (iii) use of associated devices to simplify and speed up the experiment while preventing contamination risk and reducing human errors during the use of the cryopreservation media and methods described here. Compositions, methods, and associated devices in the invention may be used in any combination, together or solely.

The applicant, Ranan Gulhan Aktas, converts the Provisional Patent Application (No. 63/388,149) that filed on Jul. 11, 2022; to a PCT application in that invention. The inventor's previous invention might also be used for that purpose instead of the device described in that invention (PCT/TR2021/051111—Multipurpose container for biological materials and methods). Various advantages of the present invention will become apparent to those skilled in the art upon reading the following detailed description, appended claims, and upon reference to the following figures. The figures and methods are examples to show that compositions in the above-mentioned concentration ranges is very well suited for cryopreservation of the entire specimen.

### BRIEF DESCRIPTION OF THE SEVERAL VIEWS OF THE DRAWING

FIG. 1 illustrates an example of a cross sectional view of the lid (11) and base (12) of an associated device (1) in accordance with certain examples.

FIG. 2 illustrates an example of the steps of the methods using the associated device (1) in accordance with certain examples. Organoids and spheroids in a gel (a) are grown (b), frozen (b), thawed (c), and cultured again (d) in a single device (1). The user only needs to change the solution in the environment with cell culture medium (f), cryopreservation media (g), a mixture of cryopreservation media and cell culture medium (h), and cell culture medium (i), respectively.

FIG. 3A illustrates an example of a live image of 2D growing cells at low magnification after cryopreservation using the Method 1.

FIG. 3B illustrates an example of a live image of 2D growing cells at high magnification after cryopreservation using the Method 1.

FIG. 4A illustrates an example of a live image of 2D growing cells at low magnification after cryopreservation using the Method 2.

FIG. 4B illustrates an example of a live image of 2D growing cells at high magnification after cryopreservation using the Method 2.

FIG. 5 illustrates an example of a graph demonstrating comparison of live cell numbers with the control groups in 10 different specimens using Method 1 and Method 2.

FIG. 6A illustrates an example of a live image of spheroids in the gel before cryopreservation using the Method 3.

FIG. 6B illustrates an example of a live image of spheroids in the gel after cryopreservation using the Method 3.

FIG. 7 illustrates an example of a picture of a spheroid in a gel after cryopreservation using the Method 3. The spheroid was labeled with live cell membrane stain and DAPI to demonstrate the viability of the entire 3D architecture.

FIG. 8 illustrates an example of a picture of an organoid in a gel after cryopreservation using the Method 4. The organoid was labeled with live cell membrane stain and DAPI to demonstrate the viability of the entire 3D architecture.

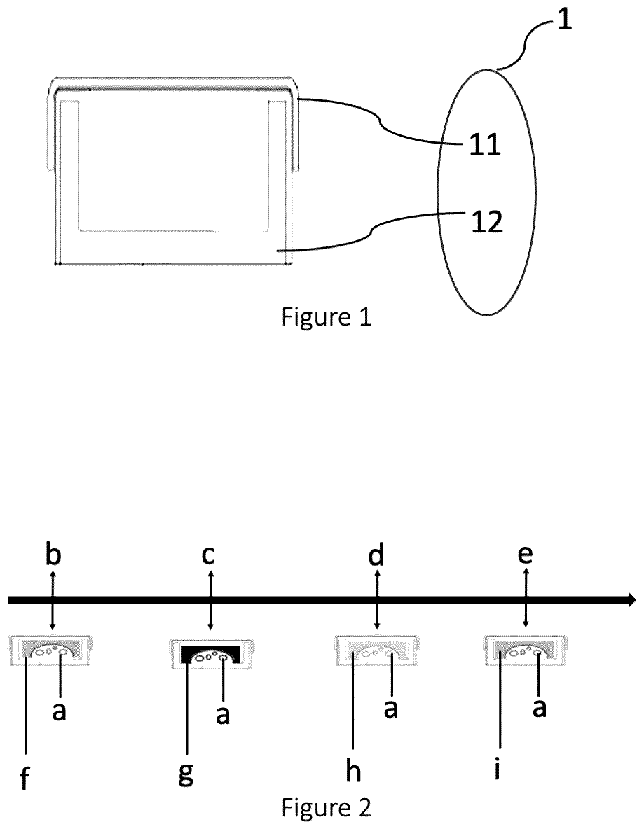

### DESCRIPTION OF REFERENCES

1 Device
11 Lid
12 Base

### DETAILED DESCRIPTION OF THE INVENTION

In this detailed description, preferred embodiments of the invention are described for a better understanding of the subject and with no limiting effect.

The present invention relates to the compositions, methods, and associated devices (1) for cryopreservation of biological specimens, e.g., organoids, spheroids, cells, cell lines, multicellular samples, living organisms, artificial or natural tissues and pathological samples, in a simplified, standardized, and straightforward way.

The composition in the invention comprises
at least one cryoprotectant agent,
at least one diluting agent,
at least one accessory agent.

In the invention, the cryoprotectant agents are selected from the group comprising glycerol/glycerin, dimethyl sulfoxide (DMSO or Me₂S), propylene glycol, ethylene glycol, polyvinylpyrrolidone, PROS, methanol, methyl acetamide, 2-methyl-2, 4-pentanediol, formamide, protein, sorbitol, triethylene glycol, polymers (Polyvinyl alcohol, PEG, etc.), sugars (Sucrose, glucose, fructose, trehalose, panose etc.), proteins (albumin, starch, etc.), and mixtures., but not limited to. The cryoprotectant agents can be used according to their purpose of use but are not limited to thereto.

In the invention, the diluting agents are selected from the group comprising basal medium (e.g., GMOPS, DMEM, RPMI medium, MEM medium, HamF-12 medium, DM-160 medium), a serum (e.g, e.g., Fetal Bovine Serum, Goat Serum), tris-citrate, ddH₂O, Saline (e.g., Ringer's Saline, Phosphate Buffered Saline, Dulbecco's Phosphate Buffered Saline, Ca and Mg free Phosphate Buffered Saline, Tris-buffered Saline), glycerin, and mixtures, but not limited to. The diluting agents can be used according to their purpose of use but are not limited to thereto. In the invention, the accessory agents are selected from the group comprising natural or synthetic extracellular matrix components, antioxidants, cell nutritional agents, albumin, steroids, organic acid polymers, fatty acids, nitroglycerin, ACE inhibitors, beta blockers, antibiotics, antimicrobial agents, antifungals, antivirals, immunosuppressive agents, non-steroidal anti-inflammatory agents, a medicament, carboxymethyl cellulose, sodium carboxymethyl cellulose, organic acid polymers, propylene glycol alginate, sodium alginate, and mixtures., but not limited to. The accessory agents can be used according to their purpose of use but are not limited to thereto.

Such a composition can comprise any of the agents listed in each group above, in a form suitable for cryopreservation methodology to a specimen, or the composition may comprise one or more agents, one or more additional diluting agents, one or more accessory agents, or some combination of these. In addition, the active ingredient may be present in the composition as a physiologically acceptable ester or salts, such as in combination with a physiologically acceptable cation or anion, as is well known in the art.

The relative amounts of the agents, the diluents, and any additional accessory agents in a composition of the invention will vary depending upon the methodology, specimen type, specimen size, microenvironment type, and condition of the specimen treated and further depending upon the route by which the compound is to be administered. For example, when the specimen size increases, like large organoids, the percentage of the cryoprotectant agents in the compound increases. In addition to that, the composition of the compound in that invention may vary according to the nature of the biological specimen. For example, each tissue or organ in the body has an extracellular matrix with a unique composition and topology. Through these physical and biochemical characteristics, the extracellular matrix generates each organ's biochemical and mechanical properties, such as its tensile and compressive strength and elasticity, and mediates protection by a buffering action that maintains extracellular homeostasis and water retention. In addition, the extracellular matrix directs the essential morphological organization and physiological function by binding growth factors and interacting with cell-surface receptors to elicit signal transduction and regulate gene transcription. The extracellular matrix's biochemical, biomechanical, protective, and organizational properties in a given tissue can vary tremendously from one tissue to another. For that reasons, the percentage of the accessory reagents including appropriate extracellular matrix components may vary according to the tissue or organ. If the thawing process is longer because of the large specimen size, the user may prefer the compound that does not contain DMSO to avoid toxic effects of DMSO. Similarly, the DMSO-free cryopreservation media including sucrose, FBS, laminin, fibrinogen, fibronectin, clastin, and collagen may be prepared for susceptible biological specimens.

In some embodiments, suited alternative concentrations for the sum of the cryoprotectant agents in the composition are in the range of preferably 6.0 w/v % to 60.0 w/v %, more preferably 10.0 w/v % to 30.0 w/v %, furthermore preferably 0.01 v/v % to 30.0 v/v %, and and particularly preferably 0.01 v/v % to 30.0 v/v % from a viewpoint of recovery rate.

In some embodiments, suited alternative concentrations for the sum of the diluting agents in the composition are in the range of preferably 0.5 v/v % to 95.0 v/v %, more preferably 1.0 v/v % to 60.0 v/v %, furthermore preferably 2.0 v/v % to 40.0 v/v %, and particularly preferably 3.0 v/v % to 30.0 v/v %.

In some embodiments, suited alternative concentrations for the sum of the accessory agents in the composition are in the range of preferably 0.01 w/v % to 80.0 w/v %, more preferably 1.0w/v % to 40.0 w/v %, furthermore preferably 2.0 w/v % to 20.0 w/v %, and particularly preferably 3.0 w/v % to 10.0 w/v %.

Other formulations suitable may include but are not limited to a powdered or granular formulation, an aqueous or oily suspension, an aqueous or oily solution, or an emulsion.

The compositions in this invention may include but are not limited to inert diluents, granulating and disintegrating agents, binding agents, and lubricating agents.

According to the cryopreservation method and the specimen, the invention might be prepared as only one compound or a kit.

The invention is also related to an associated device (1) allowing a gradual freezing and standardized reconstitution of the specimens upon thawing. The specimens in the device might be stored in −20° C., −80° C. freezers, or nitrogen tanks.

The associated device (1) in the invention comprises at least one lid (11) and at least one base (12). In addition, the secure closing system between the lid (11) and the base (12) prevents material and gas exchange during cryopreservation.

In one embodiment of the invention, the associated device (1) is made of insulating material and provides a protective environment for the specimen(s) located in container(s), e.g. cell culture plates, flasks, glass bottomed-dishes, organ-on chips, etc. The associated device (1) serves as a safe storage box inside the freezer for one or more cell culture containers. It provides cooling gradually and minimizes microbial contamination.

In another embodiment of the invention, the closing system between the lid (11) and the base (12) allows gas transition during culturing and prevents a gas exchange during the freezing and thawing process. In those embodiments, the device make it possible to culture and examine the specimen before and after cryopreservation without moving the entire structure.

The device (1) may be resistant to temperature changes between +60 and −200 C. The device (1) may comprise a scaffold or any other gel, be covered with the substance. The lid (11) and base (12) of the device (1) may comprise one or more portions with different size, shape, and materials for growing and examining the biological specimen.

In another embodiment of the invention, the shape of the base (12) may change according to the needs. For example, it can be conical, rectangular, or ‘u’ shape.

In another embodiment, the shape and the size of the device (1) change.

In another embodiment of the invention, the device (1) comprises compartments and channels of different sizes and shapes to facilitate the generation of spheroids, and organoids, and designing co-culture experiments, facilitating microfluidics studies, but not limited to thereto. The number, shape, and size of the compartments and channels may vary according to the needs.

In another embodiment of the invention, the device (1) comprises a solid support or other supportive elements.

In another embodiment of the invention, the lid (11) and/or base (12) of the device (1) is made of optical material to allow microscopic examination.

In another embodiment of the invention, the portion and/or the remaining parts of the device (1) are covered with the material to enhance growth, freeze or thawing steps.

The lid (11) and/or the base (12) can be made of from one or more of the following materials: glass, chlorotrifluoroethylene—also called aclar33c film—, polyvinylchloride, polyethersulfone, polytetrafluoroethylene, polyethylene, polyurethane, polyetherimide, polycarbonate, polysulfone, polyetheretherketone, polypropylene, polystyrene, fluoropolymer, any other polymer, any other biocompatible material, any other bioresorbable material, any thermoresistant material, any thermal insulator material, but not limited to.

The lid (11) and/or the base (12) can be covered and/or filled and/or made off with any of the following reagents, but not limited to: collagen, matrigel, laminin, fibrinogen, matrigel, hydrogel, alginate, hydroxypropyl methylcellulose phthalate, polyvinylchloride, polyethersulfone, polytetrafluoroethylene, polyethylene, polyurethane, polyetherimide, polycarbonate, polysulfone, polyetheretherketone, polycarbonate, polypropylene, fluorinated ethylene propylene, polystyrene, biocompatible material, bioresorbable material, fluoropolymer, polymers for transplantation/implantation, synthetic or natural extracellular matrix components, biocompatible materials including various extracellular matrix components, filtered membranes containing pores with a specific size for the biological material.

As used herein, “closing system” means a sealing system or a mechanism with screws or clips and a gasket that provides two positions, one tightly closed and the other allowing gas flow. The closing system serves as a an airtight seal when heated or cooled, and can contain any of the following closure system but not limited to: Continuous thread closures, lug caps, dome caps, phenolic polycone caps, ribbed closures, smooth closures, and induction cap sealing.

Throughout this specification, unless the context requires otherwise, the words ‘comprise” and “include” and variations such as “comprising” and “including” will be understood to imply the inclusion of an item or group of items, but not the exclusion of any other item or group items.

Although the associated devices (1) in the invention herein has been described with reference to embodiments, it is to be understood that these embodiments are merely illustrative of the principles and applications of the present invention. Therefore, it is to be understood that these and various other omissions, additions, and numerous modifications may be made to the illustrative embodiments. The different arrangements may be devised without departing from the spirit and scope of the invention defined by the appended claims.

In the following detailed description, reference is made to the accompanying drawings and pictures that form a part hereof, and in which are shown by way of illustration several specific embodiments of methods and devices (1). It is to be understood that other embodiments are contemplated and may be made without departing from the scope or spirit of the present disclosure.

The following detailed description, therefore, is not to be taken in a limiting sense. All scientific and technical terms used herein have meanings commonly used in art unless otherwise specified.

The definitions provided herein facilitate understanding specific terms frequently used herein and are not meant to limit the scope of the present disclosure.

As used in this specification and the appended claims, the singular forms “a,” “an”, and “the” encompass embodiments having plural referents, unless the content dictates otherwise.

As used in this specification and the appended claims, the term ‘or’ is generally employed in its sense, including “and/or unless the content dictates otherwise.

As used herein, “have,” “having,” “include,” “including,” “comprise,” “comprising,” or the like are used in their open-ended sense and generally mean “including, but not limited to.”

Any direction referred to herein, such as “bottom, “top”, “below,” “above,” and other directions and orientations are described herein for clarity in reference to the figures and are not to be limiting of an actual device (1) or system or use of the device (1) or system. Devices (1) described herein may be used in a number of directions and orientations.

Further, the present invention is not limited to the description of the embodiments above, but may be altered in various ways within the scope of the appended Claims. An embodiment based on a proper combination of technical means disclosed in different embodiments is encompassed in the technical scope of the present invention.

### Definitions

### Biological Specimen

The term “biological specimen” or “specimen” means an amount of a material that shows what the rest of the material is or should be like, e.g., a sample of biological or body tissue, organoids, and spheroids.

A biological specimen may be exemplified by, but not limited to:
1. a sample comprising cells and/or cells debris, e.g., blood sample, suspension of cloned cells, body tissue homogenate, etc.;
2. a sample comprising of intact or damaged cells of an animal body, a body tissue, an organ, a part of an organ, smear or fluid, or a sample of a tumor, e.g., a biopsy sample, a fresh tissue sample, pathological sample.
3. a sample comprising 3D growing tissue or cell or others, in a specific environment, like Matrigel, Geltrex, Cultrex, alginate, or others.
4. a sample comprising 3D growing living organisms or cells in or on scaffolds
5. a sample comprising 3D growing living organisms or cells in or on a bioresorbable material
6. a sample comprising a living organism, e.g., a sample of a medium comprising an animal, plant, bacterium, fungi, etc.;
7. a sample comprising viral particles, debris thereof, or viral products, e.g., a body Smear comprising viral nucleic acids, proteins, peptides, etc.;
8. a sample comprising reproductive cells, sperms, oocytes, embryos, etc.;
9. a sample comprising a cell organelle(s);
10. a sample comprising artificial cells or tissue blocks;
11. a sample comprising natural or recombinant biological molecules, e.g., blood plasma sample, conditioned cell culture medium, etc.
12. a sample comprising 2D or 3D growing cells and cell lines.

### Freeze

In the scope of the present application, the expression “to freeze” means to decrease the temperature of the liquid eluent and/or the biological specimen below their respective freezing point temperature to form a frozen body. In a preferred aspect of the invention's method, the cells' freezing is performed at a temperature cooling gradient range of 1 to 2° C./min. In another preferred aspect, it is performed by vitrification.

### Cryoprotectant Agent

The cryopreservation media or kit comprise one or more active cryoprotectant agents. The relative amounts of those agents will vary depending upon the technology used, specimen type, specimen size, and condition of the specimen treated.

The composition of the compounds includes various combinations of the following cryoprotectant agents according to their purpose of use but are not limited to:
Glycerol/Glycerin
Dimethyl sulfoxide (DMSO or Me2S)
Propylene glycol
Ethylene Glycol
Polyvinylpyrrolidone
PROS
Methanol
Methyl acetamide
2-methyl-2, 4-pentanediol
Formamide
Protein
Sorbitol
Triethylene glycol
Polymers (Polyvinyl alcohol, PEG, etc.)
Sugars (Sucrose, glucose, fructose, trehalose, panose etc.)
Proteins (albumin, starch, etc.)

### Diluting Agent

The diluting agent is used to reduce viscosity, enhance solubility, increase the size, and make thinner and less concentrated by admixture to optimize the methodology and improve cryopreservation. The diluting agent might be polar or non-polar, can be organic or inorganic. More than one diluting agent can be used to optimize the formula of the compound.

The composition of the compounds may include various combinations of the following diluting reagents according to their purpose of use but are not limited to:
Basal Medium (GMOPS, DMEM, RPMI medium, MEM medium, HamF-12 medium, DM-160 medium)
Serum (e.g., Fetal Bovine Serum, Goat serum).
Saline (e.g., Ringer's Saline, Phosphate Buffered Saline, Dulbecco's Phosphate Buffered Saline, Ca and Mg free Phosphate Buffered Saline, Tris-buffered Saline)
Tris-Citrate
ddH2O,
Glycerin

### Accessory Agent

Those accessory agents add value by supporting the living cells and organisms during various processes of cryopreservation.

The composition of the compounds may comprise various combinations of the following supporting accessory agents according to their purpose of use but are not limited to:
Natural or synthetic extracellular matrix components (e.g., proteoglycan, laminin, fibrinogen, fibronectin, elastin, collagen, etc.),
Antioxidants (e.g., Vitamin C),
Cell nutritional agent (e.g., Glutamine, Sodium pyruvate),
A medicament
Steroids,
Fatty cids,
Nitroglycein,
ACE inhibitors,
Beta blockers,
Gelatine,
Antibiotics,
Antimicrobial agents,
Antifungals,
Antivirals,
Immunosuppressive agents,
Non-steroidal anti-inflammatory agents

### Cryopreservation Media

As used herein, the term “cryopreservation media’ means the composition of agents listed in this invention, which will be administered to the specimen for cryopreservation using the methodology described. The formulations of the compositions described herein may be prepared by any method known or hereafter developed in the art. In general, such preparatory methods include bringing the active agents into association with a diluting agent or one or more other accessory agents.

Such a composition can comprise any of the agents listed in each group above, in a form suitable for cryopreservation methodology to a specimen, or the composition may comprise one or more agents, one or more additional diluting agents, one or more accessory agents, or some combination of these. In addition, the active ingredient may be present in the composition as a physiologically acceptable ester or salts, such as in combination with a physiologically acceptable cation or anion, as is well known in the art.

The relative amounts of the agents, the diluents, and any additional accessory agents in a compound of the invention will vary depending upon the methodology, specimen type, specimen size, microenvironment type, and condition of the specimen treated and further depending upon the route by which the compound is to be administered. For example, when the specimen size increases, like large organoids, the percentage of the cryoprotectant agents in the compound increases. In addition to that, the composition of the compound in that invention may vary according to the nature of the biological specimen. For example, each tissue or organ in the body has an extracellular matrix with a unique composition and topology. Through these physical and biochemical characteristics, the extracellular matrix generates each organ's biochemical and mechanical properties, such as its tensile and compressive strength and elasticity, and mediates protection by a buffering action that maintains extracellular homeostasis and water retention. In addition, the extracellular matrix directs the essential morphological organization and physiological function by binding growth factors and interacting with cell-surface receptors to elicit signal transduction and regulate gene transcription. The extracellular matrix's biochemical, biomechanical, protective, and organizational properties in a given tissue can vary tremendously from one tissue to another. For that reasons, the percentage of the accessory reagents including appropriate extracellular matrix components may vary according to the tissue or organ. If the thawing process is longer because of the large specimen size, the user may prefer the compound that does not contain DMSO to avoid toxic effects of DMSO. Similarly, the DMSO-free cryopreservation media including sucrose, FBS, laminin, fibrinogen, fibronectin, elastin, and collagen may be prepared for susceptible biological specimens.

In some embodiments, suited alternative concentrations for the sum of the cryoprotectant agents in the composition are in the range of preferably 0.01 v/v % to 60.0 v/v %, more preferably 10.0 v/v % to 30.0 w/v %, furthermore preferably 5.0 v/v % to 15.0 v/v %, and and particularly preferably 3.0 v/v % to 10.0 v/v % from a viewpoint of recovery rate.

In some embodiments, suited alternative concentrations for the sum of the diluting agents in the composition are in the range of preferably 0.5 v/v % to 95.0 v/v %, more preferably 1.0 v/v % to 60.0 v/v %, furthermore preferably 2.0 v/v % to 40.0 v/v %, and particularly preferably 3.0 v/v % to 30.0 v/v %.

In some embodiments, suited alternative concentrations for the sum of the accessory agents in the composition are in the range of preferably 0.01 w/v % to 80.0 w/v %, more preferably 1.0 w/v % to 40.0 w/v %, furthermore preferably 2.0 w/v % to 20.0 w/v %, and particularly preferably 3.0 w/v % to 10.0 w/v %.

Other formulations suitable may include but are not limited to a powdered or granular formulation, an aqueous or oily suspension, an aqueous or oily solution, or an emulsion.

The compositions in this invention may include but are not limited to inert diluents, granulating and disintegrating agents, binding agents, and lubricating agents.

According to the cryopreservation method and the specimen, the invention might be prepared as only one compound or a kit.

### Solid Support

The specimen may, in one embodiment, be immobilized onto a solid support, e.g., a body tissue sample immobilized on a glass or plastic slide; a cell-free sample comprising biological molecules immobilized onto a nitrocellulose membrane, a sample embedded in a biomaterial like gel, agar, alginate etc.

The invention relates to cryopreservation of the biological specimen in or on a solid support that is chemically inert under conditions described herein, i.e., the chosen support may not have any significant influence on the results.

In an embodiment, the term “Solid Support’ may be a piece of any solid water-insoluble material, e.g., a nitrocellulose membrane, glass slide, coverslip, etc. In another embodiment, the term “Solid Support’ may be a piece of any solid water-soluble material.

In another embodiment, the support may be a bioresorbable material. The biological specimen in this embodiment is immobilized on or in the support surface.

The Support may, in one embodiment, may be a one-molecular layer thick membrane or be a multimolecular layered piece of a material, e.g., plastic or glass, or a biocompatible material. The biological specimen in this embodiment is immobilized on the support surface.

In another embodiment, the Solid Support may be a three-dimensional structure, e.g., a gel, an alginate capsule, block or a mesh of fibers. In this embodiment, the specimen is immobilized within the structure.

In one embodiment, the Solid Support is a cellular membrane, e.g., the plasma membrane.

The term “immobilized’ means that a specimen or target is not movable on or within the Support or is movable to a minimal degree.

Examples of supports suitable for immobilizing the specimens include but are not limited to synthetic polymer supports, such as polystyrene, polypropylene, substituted polystyrene, e.g., aminated or carboxylated polystyrene; polyacrylamides; polyamides; polyvinylchloride; glass; agarose; nitrocellulose: nylon; polyvinylidene difluoride; surface-modified nylon, personal, hydrogels, biodegradable materials, bioresorbable materials, etc.

In other embodiment, a specimen may be itself solid, e.g., a specimen of non-fixed solid tissue (i.e., vibratome sections). In this embodiment, the specimen itself may be accounted as Solid Support comprising an immobilized target.

### Device

As used herein, the term “device’ means the container the user can freeze, store in the freezer or nitrogen tank, and then thaw the biological specimen.

In another embodiment, the term “device’ means the single container the user can freeze, store in the freezer, thaw, grow, and examine the entire specimen.

### Gel

As used herein, the term “gel’ means the environment in which the 3D growing biological specimen is located. For example, it might be a commercially available product like Matrigel, geltrex, cultrex, alginate or any other natural or synthetic product that is compatible with the growth of the biological specimen.

### Incubate

As used herein, the term “incubate’ means maintaining the biological specimen in the desired solution and temperature for a concrete period of time.

### Examples of Methods for Cryopreservation

### Method 1: Cryopreservation of 2D-Growing Cells Using Cryopreservation Media with DMSO and Extracellular Matrix Components

The cells grown on a culture dish are frozen using a composition including DMSO, FBS, laminin, fibrinogen, fibronectin, elastin, and collagen in the device (1).

### Freezing the Specimen

1. Warm up the cryopreservation media to 37° C.
2. Aspirate the cell culture medium gently.
3. Add cryopreservation media to the environment gently.
4. Seal the cell-culture dish with parafilm to prevent gas and air flow during the next steps.
5. Replace the cell-culture dish in the device (1).
6. Close the device's (1) lid (11) tightly, place the device (1) at −20° C. for 2 hours.
7. Place the device (1) at −80° C. overnight.
   The device (1) can be kept at −80° C. for six months or placed in liquid nitrogen.

### Thawing the Specimen

1. Take out the glass-bottomed dish from the device (1) in the freezer/nitrogen tank.
2. Add 1 volume of warm cell culture medium to the freezing medium (The ratio should be 1:1)
3. Place the cell-culture dish into an incubator at 37° C. Incubate 5 minute.
4. Add 1 more volume of warm cell culture medium to the mixture of freezing medium cell culture medium gently. The ratio should be 2:1. Incubate 5 minutes.
5. Aspirate the medium and cryoprotectant mixture.
6. Add 1 volume of warm cell culture medium gently and continue culturing the specimen.

### Method 2: Cryopreservation of 2D Growing Cells with DMSO-Free Cryopreservation Media Including Type I Collagen

The cells grown on a cell culture dish are frozen using a composition including sucrose, FBS, and Type I Collagen in the device (1).

### Freezing the Specimen

1. Warm up the cryopreservation media to 37° C.
2. Aspirate the cell culture medium gently.
3. Add cryopreservation media to the environment gently.
4. Seal the cell culture dish with parafilm to prevent gas and air flow during the next steps.
5. Replace the cell culture dish in the device (1).
6. Close the device's (1) lid (11) tightly, place the device (1) at −20° C. for 2 hours.
7. Place the device (1) at −80° C. overnight.
   The device (1) can be kept at −80° C. for six months or placed in liquid nitrogen.

### Thawing the Specimen

1. Take out the cell culture dish from the device (1) in the freezer/nitrogen tank.
2. Add 1 volume of warm cell culture medium to the freezing medium (The ratio should be 1:1)
3. Place the cell culture dish in an incubator at 37° C. Incubate 5 minute.
4. Add 1 more volume of warm cell culture medium to the mixture of freezing medium cell culture medium gently. The ratio should be 2:1. Incubate 5 minutes.
5. Aspirate the medium and cryoprotectant mixture.
6. Add 1 volume of warm cell culture medium gently and continue culturing the specimen.

### Method 3: Cryopreservation of Organoids or Spheroids Using the Cryopreservation Media containing DMSO and Extracellular Matrix Components

The organoids or spheroids are grown in a gel, like Matrigel, geltrex, or cultrex, located in the biocompatible and sterile base (12) of the device (1). The specimens grown in the device (1) are frozen in the composition including DMSO, FBS, laminin, fibrinogen, fibronectin, elastin, and collagen. The freezing and thawing steps are described below.

### Freezing the Specimen

1. Warm up cryopreservation media up to 37° C.
2. Aspirate the cell culture medium gently.
3. Add cryopreservation media to the environment gently.
4. Close the device's (1) lid (11) tightly and place it at −20° C. for 2 hours.
5. Place the device (1) in −80° C. overnight.
   The specimen in the device (1) can be kept at −80° C. for six months or placed in liquid nitrogen for longer term.

### Thawing the Specimen

1. Take out the device (1) from the freezer/nitrogen tank (1).
2. Add 1 volume of warm cell culture medium to the freezing medium. The ratio should be 1:1. Incubate 10 minute in an incubator at 37° C.
3. Add 1 more volume of warm cell culture medium to the freezing medium. The ratio should be 2:1. Incubate 10 minute in an incubator at 37° C.
4. Aspirate the medium and cryoprotectant mixture gently.
5. Add 1 volume of warm cell culture medium
6. Close the lid (11) loosely to allow gas and air flow and continue culturing the specimen in an incubator at 37° C.

### Method 4: Cryopreservation of Organoids or Spheroids with DMSO-Free Cryopreservation Media Containing Extracellular Matrix Components

The organoids or spheroids are grown in a gel, like Matrigel, geltrex, or cultrex, located in the biocompatible and sterile base (12) of the device (1). The specimens in the device (1) are frozen in the composition including sucrose, FBS, laminin, fibrinogen, fibronectin, elastin, and Type I collagen. The freezing and thawing steps are described below.

### Freezing the Specimen

1. Warm up the cryopreservation media up to 37° C.
2. Aspirate the cell culture medium gently.
3. Add cryopreservation medium to the environment gently.
4. Close the device's (1) lid (11) tightly and place it at −20° C. for 2 hours.
5. Place the device (1) in −80° C. overnight.
   The specimen in the device (1) can be kept at −80° C. for six months or placed in liquid nitrogen for longer term.

### Thawing the Specimen

1. Take out the device (1) from the freezer/nitrogen tank.
2. Add 1 volume of warm cell culture medium to the freezing medium. The ratio should be 1:1.
3. Close the lid (11) of the device loosely to allow gas flow. Incubate 15 minute in an incubator at 37° C.
4. Add 1 more volume of warm cell culture medium to the freezing medium. The ratio should be 2:1. Incubate 15 minutes in an incubator at 37° C.
5. Aspirate the medium and cryoprotectant mixture gently.
6. Add 1 volume of warm cell culture medium and continue culturing the specimen in an incubator at 37° C.

The above examples are only the preferred embodiments of the present invention; it should be pointed out that for those skilled in the art, under the premise without departing from the principles of the invention; can also make some improvements and modifications, these improvements and modifications also should be considered as protection scope of the present invention.

The methodologies and the contents of the cryopreservation media can be modified according to the biological specimen's nature and size and the environment surrounding the biological specimen. For example, for the big-size specimens, the duration of the thawing steps must be longer. If the environment's stiffness increases, the duration of each step is increased to facilitate the penetration of the media and concentration of some ingredients in the media increases.

The amount and range of the extracellular matrix components in the compositions change according to the type of the biological specimen.

In a preferred aspect of the methods of the invention above, the freezing of the biological specimen is performed at a temperature cooling gradient range of 1 to 2° C./min. In another preferred aspect, it is performed by vitrification.

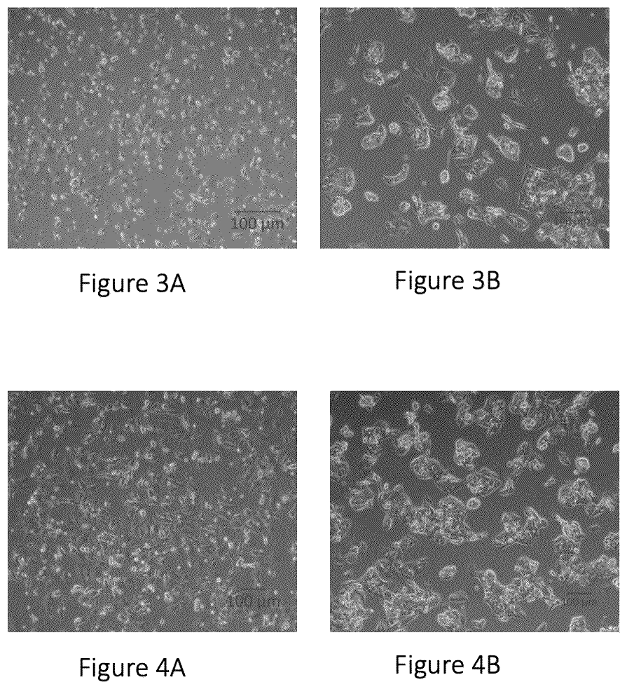

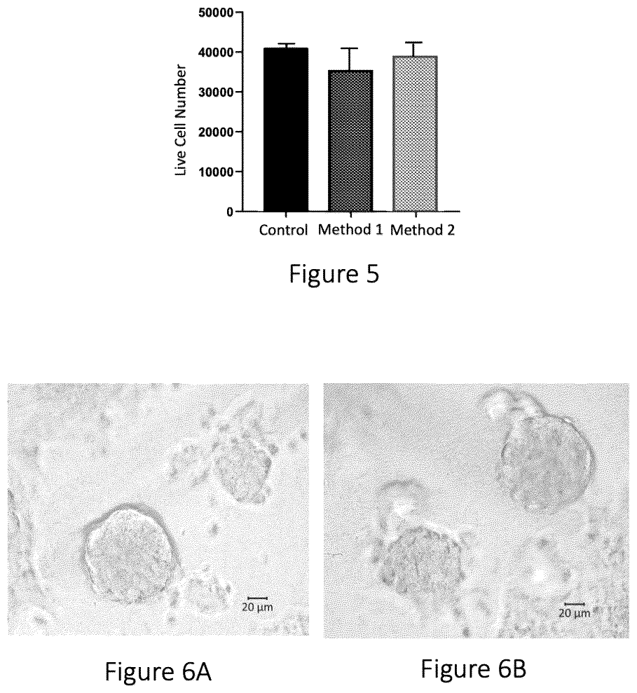

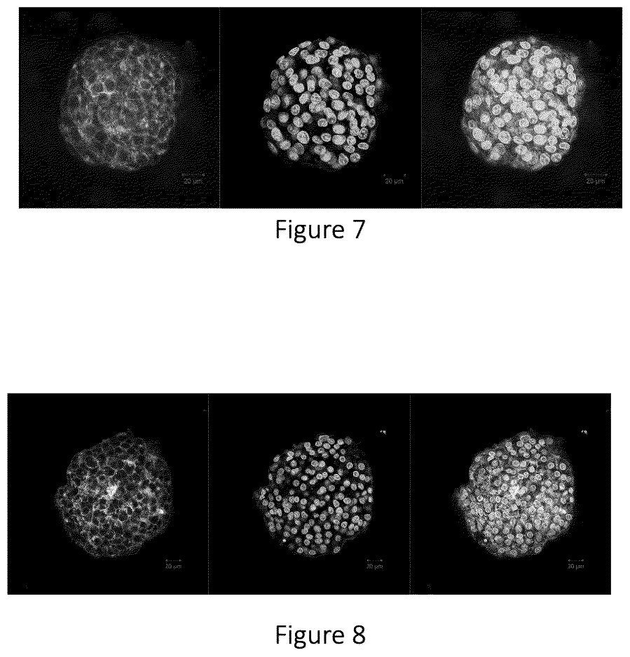

The method(s) may be performed manually, semi-, or full-automatically. The representative pictures (FIG. 3, 4, 5, 6) show the experiments' results using the methods described above.

### LITERATURE

Jarvelainen H., Sainio A., Koulu M., Wight T. N., Penttinen R. (2009). Extracellular matrix molecules: potential targets in pharmacotherapy. Pharmacol. Rev. 61, 198-223
Schaefer L., Schaefer R. M. (2010). Proteoglycans: from structural compounds to signaling molecules. Cell Tissue Res. 339, 237-246
Rozario T., DeSimone D. W. (2010). The extracellular matrix in development and morphogenesis: a dynamic view. Dev. Biol. 341, 126-140
Wise S. G., Weiss A. S. (2009). Tropoelastin. Int. J. Biochem. Cell Biol. 41, 494-497
Smith M. L., Gourdon D., Little W. C., Kubow K. E., Eguiluz R. A., Luna-Morris S., Vogel V. (2007). Force-induced unfolding of fibronectin in the extracellular matrix of living cells. PLoS Biol. 5, e268
Gosden R G, Yin H, Bodine R J, Morris G J. Character, distribution and biological implications of ice crystallization in cryopreserved rabbit ovarian tissue revealed by cryo-scanning electron microscopy. Human Reproduction. 2010;25(2):470-478
Tok O E. Demirel G. Saatci Y. Kayalar O. Akbulut Z. Aktas R G. Culturing, Culturing, Freczing. Processing, and Imaging of Entire Organoids and Spheroids While Still in a Hydrogel. J. Vis. Exp. (190), e64563, doi:10.3791/64563.2022.

1. A composition suitable for cryopreservation of biological specimens characterized by comprising
at least one cryoprotectant agent,
at least one diluting agent,
at least one accessory agent.

2. A composition according to claim 1, wherein one or more cryoprotectant agents comprising glycerol/glycerin, dimethyl sulfoxide, propylene glycol, ethylene glycol, polyvinylpyrrolidone, PROS, methanol, methyl acetamide, 2-methyl-2, 4-pentanediol, formamide, protein, sorbitol, triethylene glycol, polymers, sugars, proteins.

3. A composition according to claim 1 or 2, wherein one or more diluting agents comprising a basal medium, serum, tris-citrate, ddH₂O, glycerin, saline, any other buffered solutions.

4. A composition according to claim 1, 2 or 3, wherein one or more accessory agents comprising natural or synthetic extracellular matrix components, antioxidants, cell nutritional agents, albumin, steroids, organic acid polymers, fatty acids, nitroglycerin, ACE inhibitors, beta blockers, antibiotics, antimicrobial agents, antifungals, antivirals, immunosuppressive agents, non-steroidal anti-imflammatory agents, a medicament, carboxymethyl cellulose, sodium carboxymethyl cellulose, organic acid polymers, propylene glycol alginate, sodium alginate.

5. A method for cryopreservation of entire biological specimens in its habitat according to any one of the preceding claims, comprising steps of:
freezing,
thawing.

6. A device (1) allowing a gradual freezing and standardized reconstitution of the specimens upon thawing, characterized by comprising
at least one lid (11),
at least one base (12),
at least one closing system that provides securely sealed closing.

7. A device (1) according to claim 6, by comprising at least one closing system that provides two positions, one securely sealed closing and the other allowing gas flow.

8. A device (1) according to claim 6 or 7, characterized in that the lid (11) and/or the base (12) are made of from one or more of the following materials partly or completely: glass, chlorotrifluoroethylene-also called aclar33c film-, polyvinylchloride, polyethersulfone, polytetrafluoroethylene, polyethylene, polyurethane, polyetherimide, polycarbonate, polysulfone, polyetheretherketone, polypropylene, polystyrene, fluoropolymer, any other polymer, any other biocompatible material, any other bioresorbable material, any thermoresistant material, any thermal insulator material.

9. A device (1) according to claim 6, 7 or 8, characterized in that the lid (11) and/or the base (12) are covered and/or filled and/or made off with one or more any of the following reagents: collagen, matrigel, laminin, fibrinogen, matrigel, hydrogel, alginate, hydroxypropyl methylcellulose phthalate, polyvinylchloride, polyethersulfone, polytetrafluoroethylene, polyethylene, polyurethane, polyetherimide, polycarbonate, polysulfone, polyetheretherketone, polypropylene, polystyrene, biocompatible material, bioresorbable material, fluoropolymer, polymers for transplantation/implantation, synthetic or natural extracellular matrix components, biocompatible materials, extracellular matrix components, filtered membranes containing pores with a specific size for the biological material.

10. A device (1) according to claim 6, 7, 8 or 9, characterized in that the device (1) is designed at different sizes and/or shapes and/or contains multiple portions.

11. A device (1) according to claim 6, 7, 8, 9 or 10, characterized in that the device (1) has ultra-low attached surfaces.

12. A device (1) according to claim 6, 7, 8, 9, 10 or 11, characterized in that the lid (11) and/or the base (12) comprises scaffolds and/or solid supports.

13. A device (1) according to claim 6, 7, 8, 9, 10, 11 or 12, characterized in that the lid (11) and/or the base (12) have multiple compartments for different biological materials or different experiments.

14. A device (1) according to claim 6, 7, 8, 9, 10, 11, 12 or 13, characterized in that lid (11) and/or base (12) have compartments and/or channels between compartments connected with layer(s) located below or above compartments and channels to provide an environment for co-culture experiments.
